(19) United States
(12) Patent Application Publication
(10) Pub. No.: US 2026/0330817 A1
(43) Pub. Date: Oct. 22, 2026

(54) USER CUSTOMIZED MEDICAL IMAGE SEGMENTATION MODEL GENERATING METHOD AND SERVER

(71) Applicant: ONCOSOFT INC., Seoul (KR)

(72) Inventors: Seong Gong MOON, Seoul (KR); Jae Hee CHUN, Seoul (KR)

(73) Assignee: ONCOSOFT INC., Seoul (KR)

(21) Appl. No.: 19/111,289

(22) PCT Filed: Jun. 18, 2024
(86) PCT No.: PCT/KR2024/008414

§ 371 Date: Mar. 12, 2025

(30) Foreign Application Priority Data
Oct. 18, 2023 (KR) 10-2023-0139861

(51) Int. Cl. G06V 10/778; G06V 10/26; G06V 10/764; G06V 10/776; G06V 10/94; G06V 20/70; G16H 30/40

(52) U.S. Cl. CPC G06V 10/7788; G06V 10/26; G06V 10/764; G06V 10/776; G06V 10/945; G06V 20/70; G16H 30/40; G06V 2201/03

(57) ABSTRACT

The present disclosure is a method performed by a processor of a medical image generating apparatus including the steps of providing a model generation interface including a segmentation model selection list to predict a target area; determining a segmentation model selected by a user through the model generation interface as a learning model; acquiring a medical image to be input to the learning model and learning data for a target area specified in the medical image; and generate a segmentation model configured to predict a different target area with a 3D medical image as an input by training the learning model based on the medical image again.

### TECHNICAL FIELD

The present disclosure relates to a user customized medical image segmentation model generating method and server.

### BACKGROUND ART

Generally, in order to determine whether a subject has a disease in a target area, medical images obtained by capturing the target area of the subject using medical imaging test (for example, X-ray, ultrasound, computed tomography (CT), angiography, positron emission tomography (PET-CT), single photon emission computed tomography (SPECT-CT), or magnetic resonance imaging (MRI) test) are used. Medical staff has been performing diagnosis by visually examining medical images to identify a target area and determining whether there is a disease in the target area (for example, presence of a tumor).

However, noises may be generated in the medical image itself due to the performance of the imaging device and the movement of the subject. If the target area (for example, an organ or a tumor existing in the organ) is identified only based on the opinion of the medical staff, there may be different opinions even for the same medical image depending on the skills or experiences of the medical staff.

Accordingly, methods for predicting a target area from the medical images using an artificial neural network model trained to predict the target area based on the medical images are being widely used.

However, prediction results of an artificial neural network model may vary depending on the directivity and the learning data used by the artificial neural network model to learn it. Accordingly, a method for generating an artificial neural network model appropriate for the intention of a medical staff who wants to use the artificial neural network model is necessary, but there is no such a method.

The background of the present disclosure is described for easier understanding of the present disclosure. It should not be understood to admit the matters described in the background of the present disclosure as prior art.

### DISCLOSURE

### Technical Problem

Therefore, a method is necessary for a user to generate a customized medical image segmentation model by training the artificial neural network model using a target area segmentation result for a medical image brought by a medical staff (hereinafter, referred to as “user”).

As a result, inventors of the present disclosure configured a method for generating a user customized artificial neural network model by learning the learning data brought by a user based on a structure of an artificial neural network model can segment a medical image.

Objects of the present disclosure are not limited to the above-mentioned objects, and other objects, which are not mentioned above, can be clearly understood by those skilled in the art from the following descriptions.

### Technical Solution

In order to achieve the objects as described above, according to an aspect of the present disclosure, a user customized medical image segmentation model generating method is provided. The method is a method performed by a processor of a medical image model generating apparatus and is configured to include the steps of providing a model generation interface including a segmentation model selection list to predict any one target area; determining any one segmentation model selected by a user through the model generation interface as a learning model; acquiring a medical image to be input to the learning model and learning data for a target area specified in the medical image; and generating a segmentation model configured to predict a different target area with a 3D medical image as an input by training the learning model based on the medical image again.

According to a feature of the present disclosure, after the step of generating a segmentation model, the method may further include a step of predicting a plurality of target areas by inputting a 3D medical image including at least two or more target areas to the generated segmentation model and a previously stored segmentation model.

According to another feature of the present disclosure, after the step of generating a segmentation model, the method may further include: a step of acquiring a prediction data based on a previously stored medical image using the generated segmentation model; and a step of verifying the generated segmentation model by comparing the prediction data and the learning data.

According to still another feature of the present disclosure, after the step of verifying the segmentation model, the method may further include a step of providing a model usage interface including a search region to specify at least one of a subject condition, a target area, and a type of a segmentation model predicting the target area.

According to still another feature of the present disclosure, the step of determining as the learning model may further include: a step of providing a learner adjusting region to adjust at least one of hyper parameters, a configuration, an image coordinate system of input data, and trained weights of the learning model, by means of the model generation interface.

According to still another feature of the present disclosure, after the step of acquiring a medical image, the method may further include a step of extracting a name for a target area matched to the learning data; and a step of labeling a name extracted based on a tag list for every previously stored target area with a new learnable name.

According to still another feature of the present disclosure, the step of generating a segmentation model may further include: a step of inputting a labeled medical image to the learning model.

According to still another feature of the present disclosure, after the step of acquiring the learning data, the method may further include a step of generating a plurality of sub volume data using the medical image where the segmentation model may be a model trained to determine a region corresponding to the target area with the plurality of sub volume data as an input.

According to still another feature of the present disclosure, the sub volume data may include data regarding a moving direction of voxels configuring the sub volume data with respect to any one axis.

In order to achieve the objects as described above, according to another aspect of the present disclosure, a user customized medical image segmentation model generating server is provided. The server is configured to provide a model generation interface including a segmentation model selection list to predict any one target area, determine any one segmentation model selected by a user through the model generation interface as a learning model, acquire a medical image to be input to the learning model and learning data for a target area specified in the medical image, and generate a segmentation model configured to predict a different target area with a 3D medical image as an input by training the learning model based on the medical image again.

Other detailed matters of the exemplary embodiments are included in the detailed description and the drawings.

### Advantageous Effects

According to the present disclosure, a medical image segmentation model generating server generates an artificial neural network model for segmenting a medical image suitable for a diagnosis directivity of a user. For example, the medical image segmentation model generating server builds a target area to be predicted may be selected in a user customized manner, among various target areas, and a medical image segmentation service system including a medical image segmentation model that is selectively trained by the user.

According to the present disclosure, the medical image segmentation model generating server automatically performs a processes for verifying learning data provided by a user through an interface for generating an artificial neural network model. Accordingly, the medical image segmentation model generating server can easily generate a user customized artificial neural network model without causing a user to perform a task for standardizing data one by one for training an artificial neural network model. For example, the medical image segmentation model generating server matches learning data arbitrarily labeled by the user to be used as input data. Accordingly, the medical image segmentation model generating server increases efficiency for generating a user customized artificial neural network model and increases accessibility for generating an artificial neural network model.

According to the present disclosure, hospitals storing various medical images can use customized medical image segmentation model through the medical image segmentation model generating server. Accordingly, a medical staff device of each hospital may easily predict a target area, such as organs and lesions, through its own medical image segmentation model.

According to the present disclosure, a position of the target area in the medical image is accurately predicted and is visually displayed to help medical staff to diagnose the target area.

According to the present disclosure, the medical image segmentation apparatus improves the prediction accuracy of the model by not only inputting only 2D images configuring the medical image to a prediction model predicting a target area, but also inputting front and rear image information of sequentially captured 2D images.

The effects according to the present disclosure are not limited to the contents exemplified above, and more various effects are included in the present disclosure.

### DESCRIPTION OF DRAWINGS

FIG. 1 is a block diagram illustrating a configuration of a system to which a medical image segmentation model according to an exemplary embodiment of the present disclosure is applied.

FIG. 2A is a block diagram illustrating a user customized medical image segmentation model generating system according to an exemplary embodiment of the present disclosure.

FIG. 2B is a schematic view for explaining a user customized medical image segmentation model generating system according to an exemplary embodiment of the present disclosure.

FIG. 3 is a block diagram illustrating a configuration of a medical staff device according to an exemplary embodiment of the present disclosure.

FIG. 4 is a block diagram illustrating a configuration of a medical image segmentation model generating server according to an exemplary embodiment of the present disclosure.

FIG. 5 is a schematic flowchart illustrating a medical image segmentation model generating method according to an exemplary embodiment of the present disclosure.

FIGS. 6A and 6B are exemplary views of a user interface screen which generates a medical image segmentation model according to an exemplary embodiment of the present disclosure.

FIGS. 7A to 7C are exemplary views of a user interface screen which generates and uses a medical image segmentation model according to an exemplary embodiment of the present disclosure.

FIG. 8 is a schematic view for explaining a method for training a medical image segmentation model according to an exemplary embodiment of the present disclosure.

### MODE FOR CARRYING OUT THE DISCLOSURE

Advantages and characteristics of the present disclosure and a method of achieving the advantages and characteristics will be clear by referring to preferable exemplary embodiments described below in detail together with the accompanying drawings. However, the present disclosure is not limited to the following exemplary embodiments but may be implemented in various different forms. The exemplary embodiments are provided only to complete the disclosure of the present disclosure and to fully provide a person having ordinary skill in the art to which the present disclosure pertains with the category of the disclosure. In the description of drawings, like reference numerals denote like components.

In this specification, the terms “have”, “may have”, “include”, or “may include” represent the presence of the characteristic (for example, a numerical value, a function, an operation, or a component such as a part”), but do not exclude the presence of additional characteristic.

In the specification, the terms “A or B”, “at least one of A or/and B”, or “at least one or more of A or/and B” may include all possible combinations of enumerated items. For example, the terms “A or B”, “at least one of A and B”, or “at least one of A or B” may refer to an example which includes (1) at least one A, (2) at least one B, or (3) all at least one A and at least one B.

Although the terms “first”, “second”, and the like, may be used herein to describe various components regardless of an order and/or importance, the components are not limited by these terms. These terms are only used to distinguish one component from another. For example, a first user device and a second user device may refer to different user devices regardless of the order or the importance. For example, without departing from the scope of the present disclosure, a first component may be referred to as a second component, and similarly, a second component may be referred to as a first component.

When a component (for example, a first component) is referred to as being “operatively or communicatively coupled with/to” or “connected to” another component (for example, a second component), it can be understood that the component is directly connected to the other element, or connected to the other element via another component (for example, a third component). In contrast, when a component (for example, a first component) is referred to as being “directly coupled with/to” or “directly connected to” another component (for example, a second component), it is understood that there may not be another component (for example, a third component) between the components.

The terms “configured to (or set to)” may be exchangeably used with “suitable for”, “having the capacity to”, “designed to”, “adapted to”, “made to”, or “capable of” depending on the situation. The terms “configured to (or set to)” may not necessarily mean only “specifically designed to” in a hardware manner. Instead, in some situations, the terms “a device configured to” may mean that the device “is capable of” something together with another device or components. For example, the terms “a processor configured (or set) to perform A, B, and C” may refer to a dedicated processor (for example, an embedded processor) configured to perform the corresponding operation or a generic-purpose processor (for example, a CPU or an application processor) which is capable of performing the operations by executing one or more software programs stored in a memory device.

The terms used in this specification are merely used to describe a specific exemplary embodiment, but do not intend to limit the scope of another exemplary embodiment. A singular form may include a plural form if there is no clearly opposite meaning in the context. Terms used herein including technical or scientific terms may have the same meaning as commonly understood by those skilled in the art. Among the terms used in this specification, terms defined in the general dictionary may be interpreted as having the same or similar meaning as the meaning in the context of the related art, but are not ideally or excessively interpreted to have formal meanings unless clearly defined in this specification. In some cases, even though the terms are defined in this specification, the terms are not interpreted to exclude the exemplary embodiments of the present specification.

The features of various exemplary embodiments of the present disclosure can be partially or entirely bonded to or combined with each other and can be interlocked and operated in technically various ways understood by those skilled in the art, and the exemplary embodiments can be carried out independently or in association with each other.

For clarity of interpretation of the present specification, terms used in the present specification will be defined below.

The term used in the present specification, a “medical image” may be a 2D image configured by a plurality of cuts (or slices) of the subject. Specifically, the medical image in the present specification may be an enhanced and non-enhanced CT images generated according to the DICOM standards. For example, the medical image may include a head and neck image including fully from a skull vertex to a lung apex, a chest image including fully from thyroid to a liver dome, an abdomen image including full lumbar vertebrae/L1 spine located 3 cm cranially from the liver dome, and a pelvic image including full ischium located 3 cm cranially from the lumbar vertebrae/Li spine.

The term used in the present specification, “segmentation model” may be a model trained to predict a region corresponding to any one target area with a 3D medical image as an input. Specifically, the segmentation model in the present specification may be a model trained to predict whether a 3D slab or cube corresponds to the target area. For example, the segmentation model may be a model trained to predict whether a plurality of sub volume data each corresponds to a target area, such as a brain, a neck, a chest, an abdomen, or a pelvis. As another example, the segmentation model may be a model trained to predict whether each of the plurality of sub volume data corresponds to any one area among a plurality of organs sequentially disposed in a part of a body of a subject. Further, as another example, the segmentation model may be a model trained to predict whether each of the plurality of volume data corresponds to an area suspected of having a disease in an organ, a bone, or a muscle. Here, the area suspected of having a disease may be an area suspected of having a tumor and the medical image may be a medical image obtained by capturing the head and neck, the chest, the abdomen, and the pelvis.

In various exemplary embodiments, the segmentation model may be configured by at least one model of a fully convolutional network (FCN) having convolutional neural network (CNN) based VGG net, R, DenseNet, and an encoder-decoder structure, a deep neural network (DNN) such as SegNet, DeconvNet, DeepLAB, V3+, or U-net, or SqueezeNet, Alexnet, ResNet18, MobileNet-v2, GoogLeNet, Resnet50, Resnet101, and Inception-v3, or may be configured by two or more ensemble models.

In various exemplary embodiments, the segmentation model may be a model trained with learning data having a different slice thickness depending on a type of the medical image. In the present disclosure, a medical image segmentation model generating server or The medical staff device performs training by resampling the slice without performing the pre-processing process for increasing or reducing the slice thickness in order to unify the slice thickness according to a reference value. Specifically, the segmentation model uses slab data with a size of 256×256×16 or cubic data with a size of 96×96×96 as sub volume data and bootstraps a different number of times according to a thickness of slice input as learning data so that the segmentation model may be trained without losing the medical image. For example, when a slice with a thickness of 3 mm is input at the beginning of the learning, the segmentation model may perform the training by laminating slices to generate sub volume data in the unit of 48 mm. When a slice with a thickness of 5 mm is input at the beginning of the next learning, the segmentation model may perform the training by laminating slices to generate sub volume data in the unit of 90 mm. Such learning may be performed as many epochs as specified.

Hereinafter, the present disclosure will be described in detail by explaining preferred exemplary embodiments of the present disclosure with reference to the accompanying drawings.

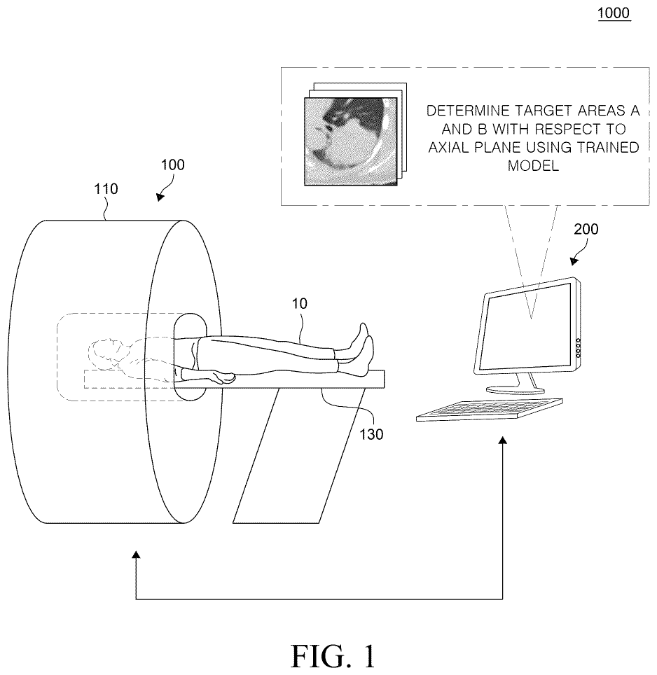

FIG. 1 is a block diagram illustrating a configuration of a system to which a medical image segmentation model according to an exemplary embodiment of the present disclosure is applied.

Referring to FIG. 1, a medical image segmentation model generating system 1000 may be a system generating a segmentation model trained to segment only a target area from the medical image. Here, segmenting the target area in the medical image segmentation model generating system 1000 can be understood as providing a region corresponding to the target area by indicating within the medical image. To this end, the medical image segmentation model generating system 1000 may include an imaging device 100 capturing a part of a body of a subject and a medical staff device 200 identifying a region corresponding to the target area, generates a medical image segmentation model to segment a region corresponding to the target area in the medical image, and diagnosing the subject.

The imaging device 100 is a device acquiring a medical image for a target area of the subject 10, such as human or animals, and may include a cylindrical bore 110 into that the subject 10 is carried and a transport device 130 where the subject 10 is seated to be carried inside. Here, the target area may include various organs, bones, and muscles, such as a brain, a neck, a chest, an abdomen, and a pelvis. The imaging device 100 may acquire a medical image for the subject 10 by irradiating X-ray that projects the subject 10, toward the subject 10.

In various exemplary embodiments, the imaging device 100 may acquire a gray scale or RGB 2D image, a still image configured by one cut, and a moving image configured by a plurality of cuts, as a medical image for predicting a region corresponding to the target area.

The medical staff device 200 may a device generating a medical image segmentation model based on a medical image captured using the imaging device 100 and displays a medical image segmentation result. For example, the medical staff device 200 may include a smart phone, a tablet personal computer (PC), a notebook, and a PC. The medical staff device 200 may newly train the medical image segmentation model based on a medical image stored in the device and a region for the target area segmented by the diagnosis of the user (medical staff). The medical staff device 200 may determine a target area with respect to any one plane using the trained model.

In various exemplary embodiments, the medical staff device 200 may be configured to install or execute a web, a mobile application, or a program provided by the medical image segmentation model generating server 300. The medical staff device 200 may display only a part corresponding to the target area in the medical image, through a web, a mobile application, or a program. The medical staff device 200 may independently performs generation of a series of medical image segmentation models performed by the medical image segmentation model generating server 300 and the arithmetic operation of the medical image segmentation model.

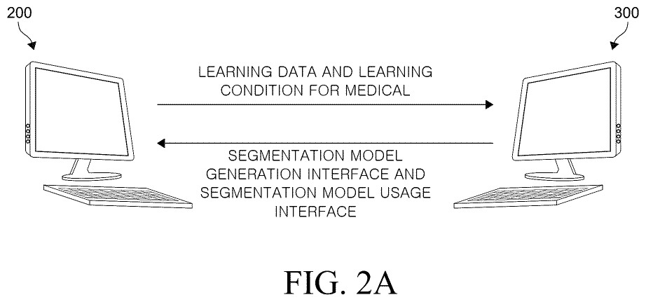

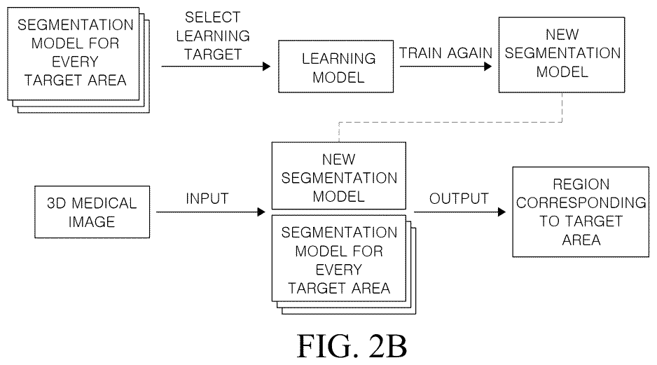

FIG. 2A is a block diagram illustrating a user customized medical image segmentation model generating system according to an exemplary embodiment of the present disclosure.

Referring to FIG. 2A, the medical image segmentation model generating system 1000 ma further include a medical image segmentation model generating server 300 generating a user customized medical image segmentation model in accordance with the request of the medical staff device 200. The medical image segmentation model generating server 300 may be a server acquiring a medical image and a learning condition from the medical staff device 200 and predicts a region corresponding to the target area from the medical image using the medical image segmentation model. For example, the medical image segmentation model generating server 300 may include a generic purpose computer, a laptop, and a data server. Here, the learning condition may include hyper parameters of a learning model, a layer configuration of a medical image segmentation model, an image coordinate system of input data corresponding to the medical image, and trained weights.

The medical image segmentation model generating server 300 performs training of the deep learning of the medical image by interacting with the medical staff device 200 to generate a user customized medical image segmentation model. To this end, the medical image segmentation model generating server 300 may provide various interfaces to the medical staff device 200 and the medical staff device 200 may generate a medical image segmentation model based on its own medical image. For example, the medical staff device 200 may display a user interface screen, such as a model generation interface to generate a medical image segmentation model, a data load interface to load a medical image captured by the imaging device 100 or stored in advance in the medical staff device 200, and a data management interface to manage learning data. As another example, the medical staff device 200 may display a model usage interface screen to display a medical image segmentation result segmented by the medical image segmentation model.

With regard to this, FIG. 2B is a schematic view for explaining a user customized medical image segmentation model generating system according to an exemplary embodiment of the present disclosure.

Referring to FIG. 2B, the medical staff device 200 may select a segmentation model to be generated in a user customized manner, among segmentation models for every target area through a model generation interface provided by the medical image segmentation model generating server 300. For example, the medical staff device 200 may select whether to train a segmentation model based on a medical image of a user for any one target area, among a brain, a neck, a chest, an abdomen, a pelvis, an optic, a heart, a breast, and bowels. As another example, the medical staff device 200 may select whether to train a segmentation model based on the medical image of the user, for any one target area, among gland thyroid, cavity oral, bone mandible, left submandibular gland, right submandibular gland, pharynx, larynx, left parotid, right parotid, left temporomandibular joint, right temporomandibular joint, left brachial plexus, and right brachial plexus in the neck. The segmentation model selected by the user may be defined as a learning model and the medical image segmentation model generating server 300 may acquire learning data for a medical image from the medical staff device 200 and train the learning model based on the learning data again to generate a new segmentation model. Here, the learning data may include a medical image, a type of the medical image, a target area specified in the medical image, a label or a tag according to the type of the target area. The new segmentation model may be a model where a structure or a weight of the existing learning model is modified according to a learning condition specified by the user.

Thereafter, when the medical image is acquired from the medical staff device 200, the medical image segmentation model generating server 300 inputs the medical image to the newly generated segmentation model and a previously stored segmentation model for each target area to output a region corresponding to the target area in the medical image. However, according to the choice of the medical staff device 200, the medical image is input only to the newly generated segmentation model to output only a region corresponding to the target area in the medical image. As described above, the medical staff device 200 may obtain a segmentation model based on the learning data provided by the medical staff device and thereafter, when a new medical image is input, may acquire a segmentation result matching its diagnostic intention using the same.

In various exemplary embodiments, the medical image segmentation model generating server 300 may provide a medical image segmentation result displaying the region corresponding to the target area in the medical image through the segmentation model usage interface screen. Here, the medical image segmentation result may be provided by emphasizing and displaying a region corresponding to the target area in each of the plurality of slices, with a different color, with respect to a plane in any one of axial, coronal, and sagittal directions.

In the meantime, in the present disclosure, the medical image segmentation model generated by the medical image segmentation model generating server 300 does not predict a region corresponding to the target area in each of a plurality of 2D slices configuring the medical image, but predicts a region corresponding to the target area in 3D volume data. Specifically, the medical image segmentation model generating server 300 may generate a plurality of sub volume data using the plurality of slices and predict a region corresponding to the target area based on the sub volume data. Here, the sub volume data may be generated by laminating the plurality of slices configuring the medical image at a predetermined height and segmenting the plurality of slices in a direction perpendicular to a plane of the slice. For example, the sub volume data may be slab data with a size of 256×256×16 and as another example, the sub volume data may be cubic data with a size of 96×96×96. Further, voxels configuring the sub volume data may have sizes of 2×2×3 mm³ or 0.7×0.7×1.0 mm³. Further, Voxels may have various sizes within the range of 0.7×0.7×1.0 mm³ to 2×2×3 mm³.

In the present disclosure, a plurality of sub volume data may include data regarding a moving direction of voxels configuring the sub volume data with respect to any one axis and increase the segmentation accuracy of the segmentation model, more than the prediction of the target area in the unit of slices. For example, when the sub volume data is slab data, each sub volume data may include data regarding the moving direction of the voxels in a direction perpendicular to the horizontal plane (axial). As another example, when the sub volume data is cubic data, each sub volume data may include data regarding the moving direction of the voxels in at least one of axial, coronal, and sagittal directions.

Accordingly, the medical image segmentation model generating server 300 may determine a region corresponding to the target area from the plurality of sub volume data, by inputting the plurality of sub volume data to a segmentation model trained to predict any one target area with a 3D medical image as an input. In other words, the medical image segmentation model generating server 300 may predict whether each of the plurality of sub volume data corresponds to the target area. For example, the medical image segmentation model generating server 300 may predict whether each of the plurality of sub volume data corresponds to a target area, such as a brain, a neck, a chest, an abdomen, or a pelvis.

In various exemplary embodiments, the medical image segmentation model generating server 300 may include a plurality of segmentation models predicting a specific region of each of the brain, the neck, the chest, the abdomen, and the pelvis and group the medical images for every target area to use any one segmentation model, among the plurality of segmentation models.

Until now, the medical image segmentation model generating system 1000 according to the exemplary embodiment of the present disclosure has been described. According to the present disclosure, the medical image segmentation model generating system 1000 generates a medical image segmentation model based on the medical image stored in the medical staff device 200 again and accordingly segments the medical image, thereby increasing medical staff's usage convenience and accessibility to the system and help the medical staff to diagnose the target area.

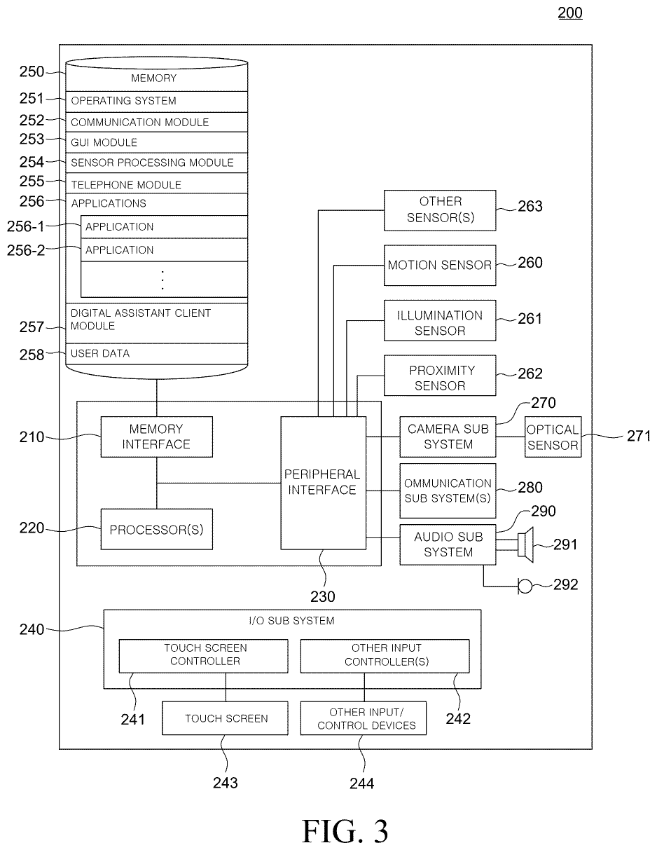

Hereinafter, a medical staff device 200 to generate a user customized medical image segmentation model will be described with reference to FIG. 3.

FIG. 3 is a block diagram illustrating a configuration of a medical staff device according to an exemplary embodiment of the present disclosure.

Referring to FIG. 3, the medical staff device 200 may include a memory interface 210, one or more processors 220, and a peripheral interface 230. Various components in the medical staff device 200 may be connected by one or more communication buses or signal lines.

The memory interface 210 is connected to a memory 250 to transmit various data to the processor 220. Here, the memory 250 may include at least one type of storing media of a flash memory type, a hard disk type, a multimedia card micro type, a card type memory (for example, an SD or XD memory), a RAM, an SRAM, a ROM, an EEPROM, a PROM, a network storage, a cloud, and a block chain database.

In various exemplary embodiments, the memory 250 may store a web/app application or a program for segmenting only a region corresponding to the target area from the medical image using a user customized medical image segmentation model. Further, the memory 250 may store subject's identification information (for example, age, gender, whether to have a disease), a medical image of the subject, a region corresponding to the target area in the medical image, a label or a tag of the medical image.

In various exemplary embodiments, the memory 250 may store at least one of an operating system 251, a communication module 252, a graphic user interface module (GUI) 253, a sensor processing module 254, a telephone module 255, and an application module 256. Specifically, the operating system 251 may include an instruction for processing a basic system service and an instruction for performing hardware tasks. The communication module 252 may communicate with at least one of one or more other devices, computers, and servers. The graphic user interface module (GUI) 253 may process a graphic user interface. The sensor processing module 254 may process sensor-related functions (for example, to process a received voice input using one or more microphones 292). The telephone module 255 may process telephone-related functions. The application module 256 may perform various functions of the user application, such as electronic messaging, web browsing, media processing, searching, imaging, or other processing functions. Further, the medical staff device 200 may store one or more software applications 256-1 and 256-2 (for example, a medical image segmentation model generating application) related to any one type of service in the memory 250.

In various exemplary embodiments, the memory 250 may store a digital assistant client module 257 (hereinafter, simply referred to as a DA client module) and accordingly, may store instructions for performing a function of the client of the digital assistant and various user data 258 (for example, user customized vocabulary data, preference data, or other data such as a user's electronic address book).

In the meantime, the DA client module 257 may acquire voice input, text input, touch input and/or gesture input of the user by means of various user interfaces (for example, I/O sub system 240) equipped in the medical staff device 200.

Further, the DA client module 257 may output audio-visual or tactile data. For example, the DA client module 257 may output data formed of a combination of at least two or more of voice, sound, a notice, a text message, a menu, a graphic, a video, an animation, and a vibration. Further, the DA client module 257 may communicate with a digital assistant server (not illustrated) using a communication sub system 280.

In various exemplary embodiments, the DA client module 257 may collect additional information about the surrounding environment of the medical staff device 200 from various sensors, sub systems, and peripheral devices to configure a context associated with the user input. For example, the DA client module 257 may infer the intention of the user by providing context information to the digital assistant server together with the user input. Here, the context information may be accompanied by the user input may include sensor information, such as light, ambient noises, ambient temperature, an image of the surrounding environment, and a video. As another example, the context information may include a physical state (for example, a device alignment, a device position, a device temperature, a power level, a speed, an acceleration, a motion pattern, or a cellular signal intensity) of the medical staff device 200. As another example, the context information may include information related to a software state of the medical staff device 200 (for example, a process being executed in the medical staff device 200, installed program, past and present network activities, a background service, an error log, or resource usage).

In various exemplary embodiments, the memory 250 may include added or deleted instructions. Moreover, the medical staff device 200 may also include an additional configuration, in addition to the configuration illustrated in FIG. 3 or exclude some configurations.

The processor 220 may control an overall operation of the medical staff device 200 and perform various commands to generate a medical image segmentation model, display a region corresponding to the target area in the medical image through a medical staff or implement a user interface to identify a prediction result provided from the segmentation model, by driving the application or the program stored in the memory 250.

The processor 220 may correspond to an arithmetic device such as a central processing unit (CPU) or an application processor (AP). Further, the processor 220 may be implemented as an integrated chip (IC) such as a system of chip (SoC) in which various arithmetic devices which perform machine learning, such as a neural processing unit (NPU), are integrated.

In various exemplary embodiments, the processor 220 may generate a medical image segmentation model through an application or a program provided from the medical image segmentation model generating server 300 and output a result displaying a region corresponding to a target area in a medical image newly obtained using the generated medical image segmentation model.

In various exemplary embodiments, the processor 220 may provide a model generation interface including a segmentation model selection list for predicting any one target area to the user, determine any one segmentation model selected by the user through the model generation interface as a learning model, acquire a medical image to be input to the learning model and learning data for a target area matched to the medical image, and generate a segmentation model configured to predict a different target area with a 3D medical image as an input by training the learning model based on the medical image again. Specifically, the processor 220 may generate a plurality of sub volume data using the medical image and use the plurality of sub volume data as input data.

The peripheral interface 230 is connected to various sensors, sub systems, and peripheral devices to provide data to allow the medical staff device 200 to perform various functions. Here, when the medical staff device 200 performs any function, it is understood that the function is performed by the processor 220.

The peripheral interface 230 may receive data from a motion sensor 260, an illumination sensor (an optical sensor) 261, and a proximity sensor 262 and by doing this, the medical staff device 200 may perform alignment, light, and proximity sensing functions. As another example, the peripheral interface 230 may be provided with data from other sensors 263 (a positioning system-GPS receiver, a temperature sensor, or a biometric sensor), and by doing this, the medical staff device 200 may perform functions related to the other sensors 263.

In various exemplary embodiments, the medical staff device 200 may include a camera sub system 270 connected to the peripheral interface 230 and an optical sensor 271 connected thereto and by doing this, the medical staff device 200 may perform various photographing functions such as taking a picture or recording a video clip.

In various exemplary embodiments, the medical staff device 200 may include a communication sub system 280 connected to the peripheral interface 230. The communication sub system 280 is configured by one or more wired/wireless networks and may include various communication ports, a wireless frequency transceiver, and an optical transceiver.

In various exemplary embodiments, the medical staff device 200 includes an audio sub system 290 connected to the peripheral interface 230 and the audio sub system 290 includes one or more speakers 291 and one or more microphones 292 so that the medical staff device 200 may perform voice-operated functions, such as voice recognition, voice duplication, digital recording, and telephone functions.

In various exemplary embodiments, the medical staff device 200 may include an I/O sub system 240 connected to the peripheral interface 230. For example, the I/O sub system 240 may control the touch screen 243 included in the medical staff device 200 by means of a touch screen controller 241.

For example, the touch screen controller 241 may use any one of a plurality of touch sensing techniques such as a capacitive type, a resistive type, an infrared type, a surface acoustic wave technology, or a proximity sensor array to detect contact and movement of the user or stopping of contact and movement. As another example, the I/O sub system 240 may control the other input/control device 244 included in the medical staff device 200 by means of other input controller(s) 242. As an example, other input controller(s) 242 may control one or more buttons, rocker switches, thumb-wheels, infrared ports, USB ports, and pointer devices such as a stylus.

Until now, the medical staff device 200 according to the exemplary embodiment of the present disclosure has been described. According to the present disclosure, a target area, for example, a position and a size of an organ, a bone, or a tumor, to be diagnosed from the medical image, may be identified in accordance with the medical staff's intention by generating a medical staff's unique medical image segmentation model using the medical staff device 200 and thus the health condition of the subject may be accurately diagnosed.

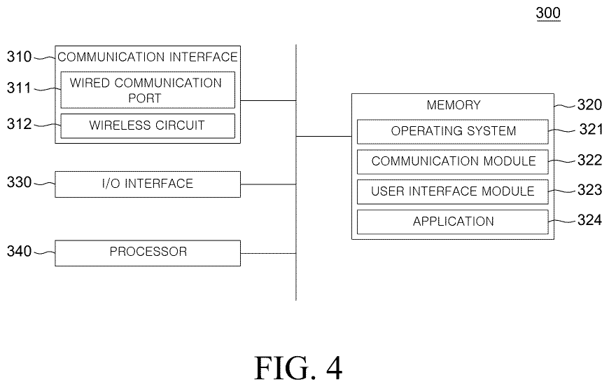

Hereinafter, a medical image segmentation model generating server that generates and verifies a medical image segmentation model and then provides a service will be described with reference to FIG. 4.

FIG. 4 is a block diagram illustrating a configuration of a medical image segmentation model generating server according to an exemplary embodiment of the present disclosure.

Referring to FIG. 4, the medical image segmentation model generating server 300 may include a communication interface 310, a memory 320, an I/O interface 330, and a processor 340 and each configuration may communicate with each other via one or more communication buses or signal lines.

The communication interface 310 is connected to the imaging device 100 and the medical staff device 200 via a wired/wireless communication network to exchange data. For example, the communication interface 310 may receive a medical image from the imaging device 100 or the medical staff device 200 and receive a request to generate a medical image segmentation model based on the medical image from the medical staff device 200. As another example, the communication interface 310 may transmit an interface for generating a medical image segmentation model to the medical staff device 200 and provide a medical image segmentation model trained again based on a medical image and learning data including a medical image.

In the meantime, the communication interface 310 that enables the transmission/reception of the data includes a wired communication port 311 and a wireless circuit 312 and the wired communication port 311 may include one or more wired interfaces, such as Ethernet, universal serial bus (USB), and fire wire. Further, the wireless circuit 312 may transmit and receive data with external devices by an RF signal or an optical signal. In addition, the wireless communication may use at least one of a plurality of communication standards, protocols, and technologies, such as GSM, EDGE, CDMA, TDMA, Bluetooth, Wi-Fi, VoIP, Wi-Max, or other arbitrary appropriate communication protocols.

The memory 320 may store various data used in the medical image segmentation model generating server 300. For example, the memory 320 may store configurations and learning data of a segmentation model trained to predict a region corresponding to any one target area with identification information of the imaging device 100 and the medical staff device 200, a medical image provided from the medical staff device 200, a tag list for each target area, and a 3D medical image as inputs, a newly trained segmentation model, and a classification model trained to segment a type of a target area with a medical image as an input.

In various exemplary embodiments, the memory 320 may include a volatile or nonvolatile recording medium which may store various data, commands, and information. For example, the memory 320 may include at least one type of storage media of a flash memory type, a hard disk type, a multimedia card micro type, a card type memory (for example, an SD or XD memory), a RAM, an SRAM, a ROM, an EEPROM, a PROM, a network storage, a cloud, and a block chain database.

In various exemplary embodiments, the memory 320 may store a configuration of at least one of an operating system 321, a communication module 322, a user interface module 323, and one or more applications 324.

The operating system 321 (for example, an embedded operating system, such as LINUX, UNIX, MAC OS, WINDOWS, VxWorks) may include various software components and drivers which control and manage a general system task (for example, memory management, storage device control, or power management) and support communication between various hardware, firmware, and software components.

The communication module 322 may support communication with other devices through the communication interface 310. The communication module 322 may include various software components for processing data received by a wired communication port 311 or a wireless circuit 312 of the communication interface 310.

The user interface module 323 may receive a request or an input of the user from a keyboard, a touch screen, a mouse, or a microphone via the I/O interface 330 and provide the user interface on the display.

The application 324 may include a program or a module configured to be executed by one or more processors 340. Here, the application for generation and training of a medical image segmentation model and an arithmetic operation of a segmentation model may be implemented on a server farm.

The I/O interface 330 may connect at least one of input/output devices (not illustrated) of the medical image segmentation model generating server 300, such as a display, a keyboard, a touch screen, and a microphone, to the user interface module 323. The I/O interface 330 may receive the user input (for example, voice input, keyboard input, or touch input) together with the user interface module 323 and process a command in accordance with the received input.

The processor 340 may be connected to the communication interface 310, the memory 320, and the I/O interface 330 to control an overall operation of the medical image segmentation model generating server 300, train the segmentation model through the application or the program stored in the memory 320, and perform various commands to segment a target area in the medical image.

The processor 340 may correspond to an arithmetic device such as a central processing unit (CPU) or an application processor (AP). Further, the processor 340 may be implemented as an integrated chip (IC) such as a system of chip (SoC) in which various arithmetic devices are integrated. Further, the processor 340 may include a module for calculating an artificial neural network model like a neural processing unit (NPU).

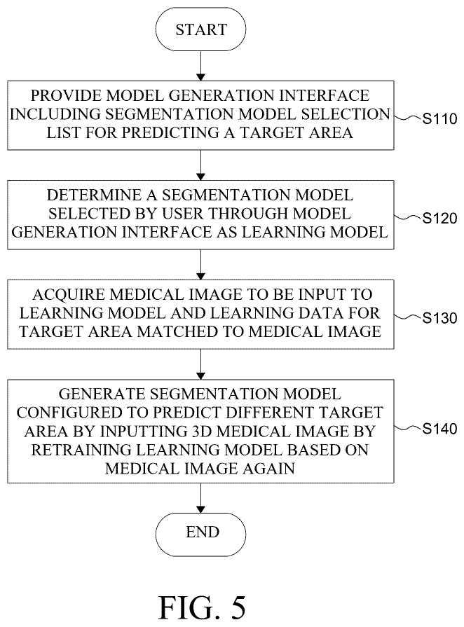

Hereinafter, a method for generating a medical image segmentation model by a processor 340 of the medical image segmentation model generating server 300 will be described with reference to FIGS. 5 to 7C.

FIG. 5 is a schematic flowchart illustrating a medical image segmentation model generating method according to an exemplary embodiment of the present disclosure.

Referring to FIG. 5, the processor 340 may provide a model generation interface including a segmentation model selection list for predicting a target area in step S110. Specifically, the processor 340 may provide a model generation interface to select a target area to be newly learned and a corresponding segmentation model to the medical staff device 200. The segmentation model selection list may include medical image segmentation models trained in advance by a manager or a user (medical staff) and include data about a name of the segmentation model, a predictable target area (for example, neck, abdomen, brain, or breast), a type (dimension) of input data (for example, 3D slab or cubic data), a size (model shape, for example, 384×384×16×4, 96×96×96×17). Furthermore, the segmentation model does not predict a target area from a medical image in the units of 2D slices, but predicts a target area using a medical image in 3D units.

After the step S110, the processor 340 may determine a segmentation model selected by the user through the model generation interface as a learning model in step S120. Specifically, when a segmentation model is selected from the segmentation model selection list, the processor 340 may provide a learner adjusting region that changes a structure of the segmentation model and input data using the model generation interface.

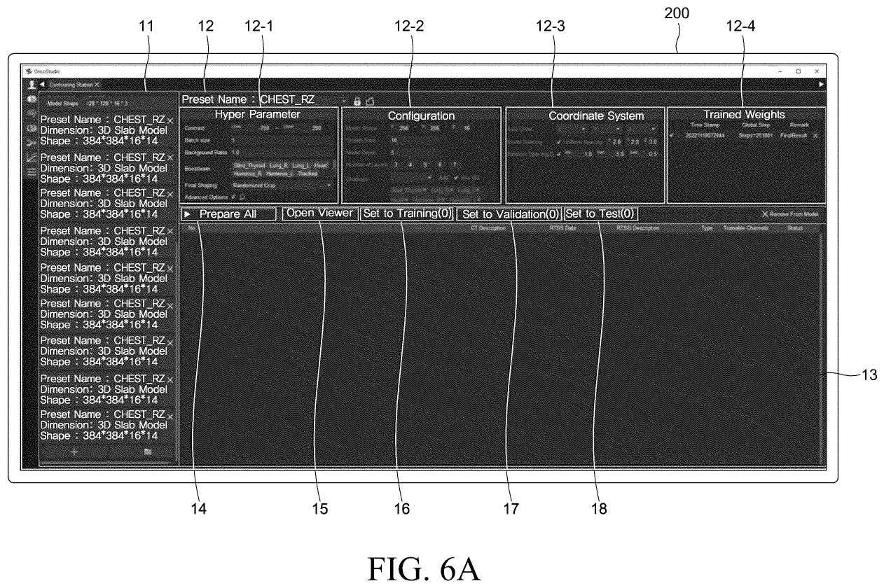

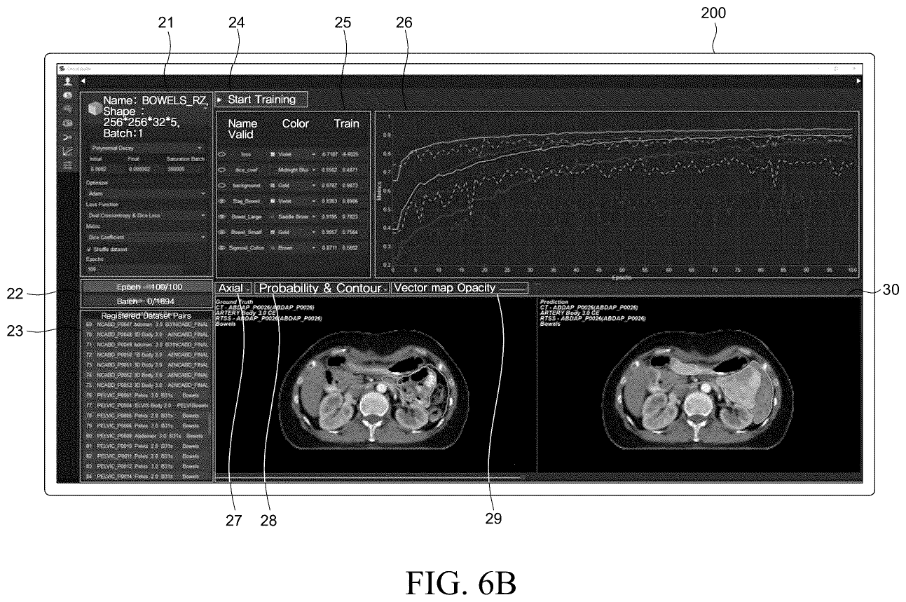

With regard to this, FIGS. 6A and 6B are exemplary views of a user interface screen which generates a medical image segmentation model according to an exemplary embodiment of the present disclosure.

Referring to FIG. 6A, the processor 340 may provide a model generation interface including a medical image segmentation model list 11 trained by a manager or a user to the medical staff device 200. When any one segmentation model is selected from the medical image segmentation model list 11 as a learning model, by the user, the processor 340 may provide a learner adjusting region 12 changing a structure of the learning model and specific information of the input data through the model generation interface. For example, the learner adjusting region 12 of the model generation interface may include a region 12-1 for setting hyper parameters, such as a contrast, a batch size, a background ratio, a boostlearn (for example, Lung_R, Lung_L, Heart, Humerus_R, Humerus_L, Trachea), or final shaping (for example, normalize, randomized shaping), a region 12-2 for setting a configuration of the learning model, such as a model shape, a growth rate, a model depth, a number of layers, and a number of channels, a region 12-3 for setting an image coordinate system of a medical image that is input data, and a region 12-4 for setting trained weights.

Referring to FIG. 5 again, the processor 340 may acquire a medical image to be input to the learning model and learning data for a target area matched to the medical image in step S130. Here, the learning data may include a medical image (for example, enhanced and non-enhanced computed tomographic (CT) images), a type of medical image (for example, a 3D slab or cubic data), a target area specified in the medical image (for example, an image marked in the medical image), a label or a tag according to a type of the target area (for example, a brain, a neck, a chest, an abdomen, or a pelvis).

With regard to this, referring to FIG. 6A again, the processor 340 may provide a model generation interface further including a region 13 where a list for medical images acquired in step S130 is displayed to the medical staff device 200. Further, the model generation interface may include a graphic object 14 for pre-processing a medical image, a graphic object 15 for displaying a medical image, a graphic object 16 for selecting a medical image to be used for learning, a graphic object 16 for verifying a medical image to be used for learning, a graphic object 17 for selecting a medical image that serves as a criterion for verifying a segmentation model that is newly generated after the learning, and a graphic object 18 for selecting a medical image that serves as a criterion for evaluating a segmentation model that is newly generated after the learning.

In the meantime, even though the medical image acquired from the medical staff device 200 is an image for the same target area, the medical image may be matched with a different term, depending on the medical staff. Accordingly, when the processor 340 acquires learning data, the processor 340 may extract a name for the target area matched to the learning data and label the extracted name based on a previously stored tag list for each of target areas as a learnable new name. As described above, the processor 340 may label a medical image included in the learning data with a tag stored in advance in the memory 320 and input the labeled medical image to the learning model to increase the efficiency of the learning of the classification model. If the name does not match, the processor 340 may group a plurality of slices configuring the medical image, for each target area using the classification model. Specifically, the processor 340 may input the acquired medical image to the classification model trained to segment a type of the target area with the medical image as an input to classify at least one target area, among the brain, the neck, the chest, the abdomen, and the pelvis.

Further, the segmentation model generated according to the present disclosure has 3D volume data as an input so that the processor 340 may generate a plurality of sub volume data using the medical image. The sub volume data may be generated by laminating the plurality of slices configuring the medical image at a predetermined height and segmenting the plurality of slices in a direction perpendicular to a plane of the slice. For example, the sub volume data may be slab data with a size of 256×256×16 and as another example, the sub volume data may be cubic data with a size of 96×96×96. Further, voxels configuring the sub volume data may have sizes of 2×2×3 mm³ or 0.7×0.7×1.0 mm³. Further, Voxels may have various sizes within the range of 0.7×0.7×1.0 mm³ to 2×2×3 mm³. The processor 340 may generate a plurality of sub volume data with respect to an axis including any one plane, among axial, coronal, and sagittal planes.

In various exemplary embodiments, a plurality of sub volume data may include data regarding a moving direction of voxels configuring the sub volume data with respect to any one axis and increase the accuracy of the prediction result, more than the prediction of the target area in the unit of slices. For example, when the sub volume data is slab data, each sub volume data may include data regarding the moving direction of the voxels in a direction perpendicular to the horizontal plane (axial). As another example, when the sub volume data is cubic data, each sub volume data may include data regarding the moving direction of the voxels in at least one of axial, coronal, and sagittal directions.

After the step S130, the processor 340 may generate a segmentation model configured to predict a different target area by inputting a 3D medical image by retraining the learning model based on the medical image again in step S140. The processor 340 may train the segmentation model trained to output a region A as a target area with the medical image as an input to output a region A′ as a target area with the medical image as an input in accordance with the user's request. Here, the overall prediction results for the region A and the region A′ are the same, but may be different in a region adjacent to the organ and the bones. Accordingly, even though the same target area is segmented, the newly trained segmentation model may have a different region for the target area.

Referring to FIG. 6B, the processor 340 may provide a model generation interface that generates a segmentation model using the learning model and displays a prediction result obtained by the segmentation model to the medical staff device 200. The model generation interface may include an area 21 that inputs and adjusts a parameter, such as a batch size, iteration, and epoch, a region 22 that displays a learning progress situation, and a region 23 that displays learning data used for the training. In addition, the model generation interface may include a graphic object 24 for training a segmentation model and a graphic object that selects whether to visually display a prediction result. As the graphic object 24 is selected, a loss function and a scale used for the learning may be displayed in the region 25 that displays a segmentation model list to perform the training. Whenever a batch ends, a train value may be updated. The processor 340 may update a valid value once the entire learning data is completely trained, that is, whenever each epoch ends. Further, the model generation interface may include a region 26 that displays a train value and a valid value on a graph for every segmentation model as the segmentation model is trained. For example, the train value may be represented with a solid line and the valid value may be represented with a dotted line. Further, the model generation interface may include a region 30 for displaying a prediction result of the target area using the segmentation model in the medical image. Here, the model generation interface may include a graphic object 27 for selecting that cross-section becomes a reference for displaying a region corresponding to the target area, a graphic object 28 for selecting that method is used to display a region corresponding to the target area, and a graphic object 29 for setting an opacity that displays a region corresponding to the target area in the medical image. Here, the model generation interface may emphasize and display only a region corresponding to the target area in the medical image and display a prediction result for each of the plurality of sub volume data, simultaneously.

In various exemplary embodiments, the processor 340 may input a 3D medical image including at least two or more target areas to the segmentation model generated in the step S140 and a previously stored segmentation model to predict a plurality of target areas. For example, the medical image may include a head and neck image including fully from a skull vertex to a lung apex, a chest image including fully from thyroid to a liver dome, an abdomen image including full lumbar vertebrae/L1 spine located 3 cm cranially from the liver dome, and a pelvic image including full ischium located 3 cm cranially from the lumbar vertebrae/Li spine. As described above, the processor 340 inputs the 3D medical image including two or more target areas to a segmentation model generated by interaction with the user and a previously stored segmentation model to predict a plurality of target areas. However, according to the choice of the medical staff device 200, the processor 340 inputs the medical image only to the newly generated segmentation model to output only a region corresponding to the target area in the medical image.

In various exemplary embodiments, the processor 340 may verify the segmentation model before using the previously generated segmentation model. Specifically, the processor 340 may obtain prediction data based on a previously stored medical image using the generated segmentation model. The processor 340 may verify the generated segmentation model by comparing prediction data and learning data. Here, the learning data to be compared may be data specified by the user through the model generation interface. The processor 340 may determine whether a region for the target area displayed with prediction data and a region for the target area included in the learning data are the same by a predetermined ratio or more. Accordingly, when two data are the same by a predetermined ratio or more, the processor 340 may determine the generated segmentation model as an available segmentation model. If two data are not the same, the processor 340 may provide an alarm to the medical staff device 200 to train the learning model again.

In various exemplary embodiments, the processor 340 may provide a model usage interface using a previously trained segmentation model together with a segmentation model that is completely verified to the medical staff device 200. Specifically, the processor 340 may provide the model usage interface including a search region to specify at least one of a subject condition, a target area, and a type of a segmentation model predicting the target area.

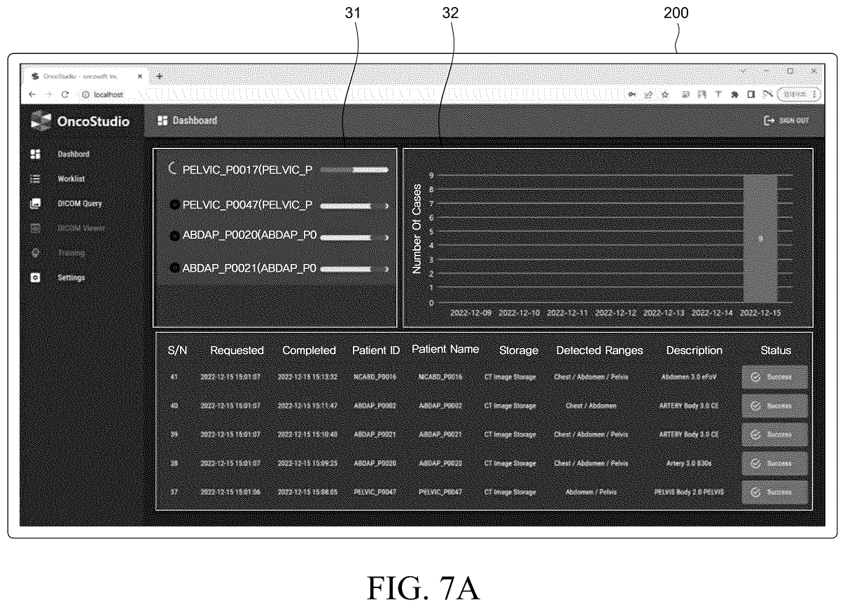

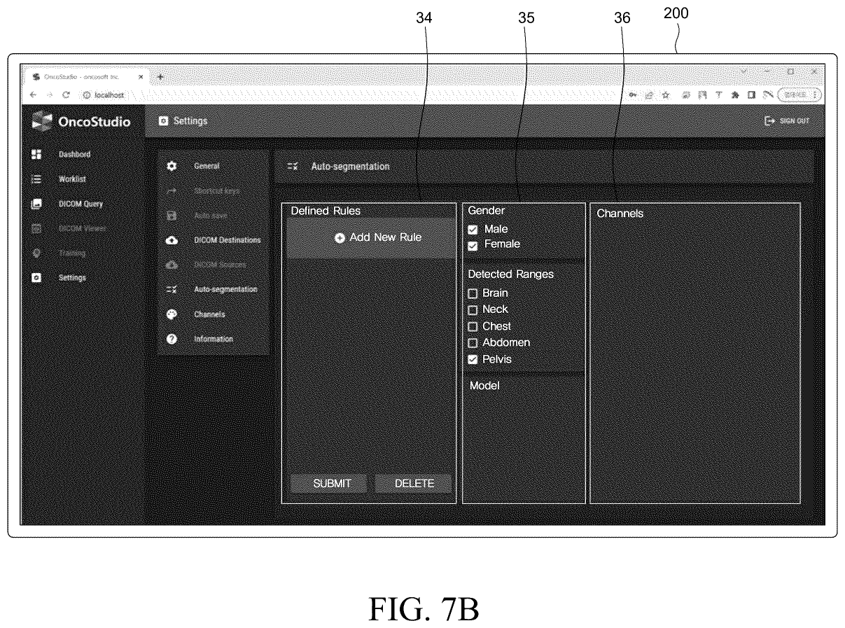

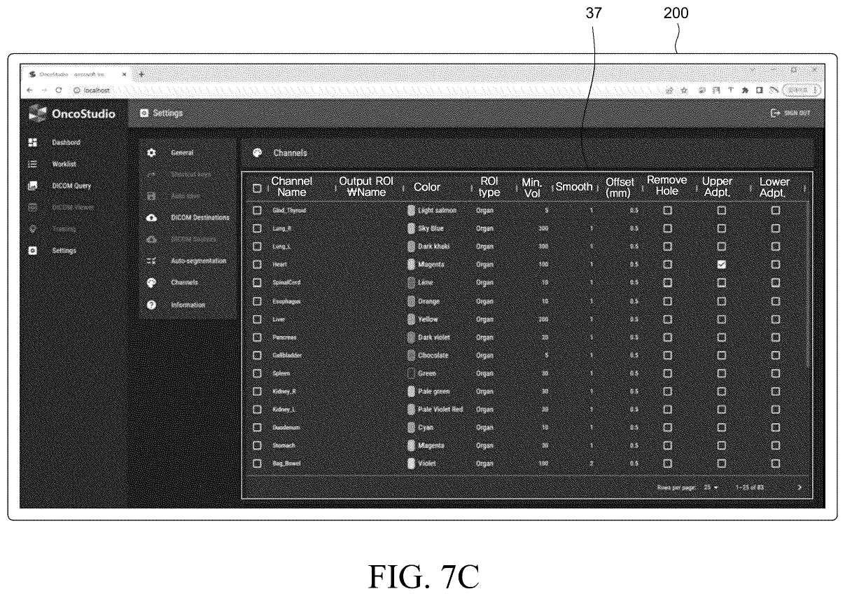

With regard to this, FIGS. 7A to 7C are exemplary views of a user interface screen which generates and uses a medical image segmentation model according to an exemplary embodiment of the present disclosure.

Referring to FIG. 7A, the processor 340 may provide a model usage interface including a region 31 where a segmentation model list being computed is displayed, a region 32 where the number of segmentation models used per day is displayed, and a region 33 where a segmentation model usage history is displayed to the medical staff device 200.

Referring to FIG. 7B, when the user wants to use the segmentation model, the processor 340 may provide a model usage interface including a region 34 where a usage rule of a segmentation model generated by the user is displayed, a region 35 for setting a gender of the subject, a body range to be segmented, and a segmentation model, and a region 36 for setting any one channel of channels included in the segmentation model to the medical staff device 200. Here, the usage rule of the segmentation model may be specified by the user when the segmentation model is generated and the user may segment and predict target areas in the medical image using a segmentation model desired by the user.

Referring to FIG. 7C, the processor 340 may provide a model usage interface including a region 37 where detailed information of a channel included in the segmentation model is displayed to the medical staff device 200 in accordance with the user's request. Specifically, the detailed information of each channel may include information about a name of the target area and a color to be displayed in the medical image, a type of the target area (for example, organs or bones).

Until now, the medical image segmentation model generating server 300 according to the exemplary embodiment of the present disclosure has been described. According to the present disclosure, the medical image segmentation model generating server 300 may generate and provide a user customized medical image segmentation model for every hospital storing a wide variety of medical images.

Hereinafter, an overall flow of the learning process of the segmentation model performed by the medical image segmentation model generating server 300 will be described.

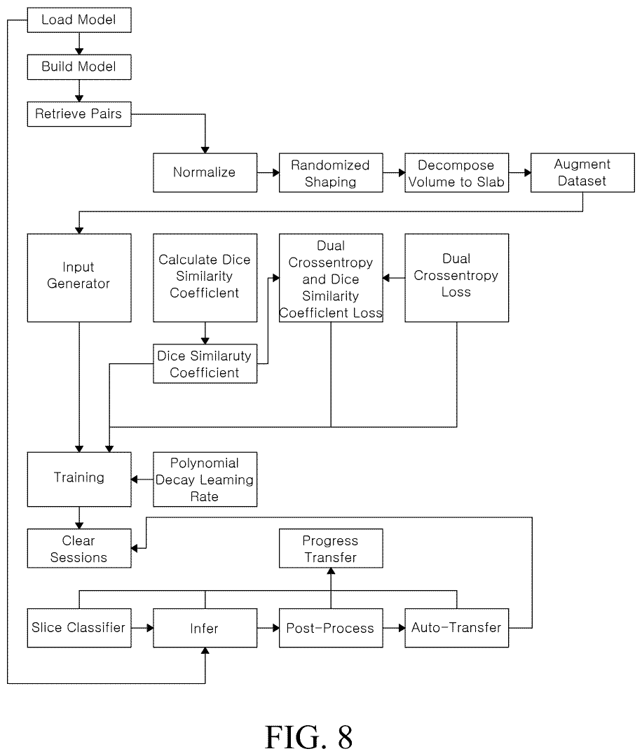

FIG. 8 is a schematic view for explaining a method for training a segmentation model according to an exemplary embodiment of the present disclosure.

Referring to FIG. 8, the medical image segmentation model generating server 300 may determine any one learning model, among a plurality of segmentation models and preprocess a plurality of slices (learning dataset) used for the learning, and then generate a plurality of sub volume data based thereon. Here, the pre-processing may correspond to a step of adjusting a size or a resolution of the medical image. The medical image segmentation model generating server 300 may input the plurality of sub volume data to perform the learning and provide an operator to correct a difference between a learning result and an actual result.

As described above, the medical image segmentation model generating server 300 may train the segmentation model again. When a prediction request for a medical image is received in accordance with a request of a manager or a medical staff, the medical image segmentation model generating server 300 may load the segmentation model to segment a region corresponding to the target area in the medical image.

Although an exemplary embodiment of the present disclosure has been described in detail with reference to the accompanying drawings, the present disclosure is not limited thereto and may be embodied in many different forms without departing from the technical concept of the present disclosure. Therefore, the exemplary embodiments of the present disclosure are provided for illustrative purposes only but not intended to limit the technical concept of the present disclosure. The scope of the technical concept of the present disclosure is not limited thereto. Therefore, it should be understood that the above-described exemplary embodiments are illustrative in all aspects and do not limit the present disclosure. The protective scope of the present disclosure should be construed based on the following claims, and all the technical concepts in the equivalent scope thereof should be construed as falling within the scope of the present disclosure.
[National R&D Project which supports this disclosure]
[Project Identification Number] 1425160412
[Project Number] S3033240
[Government Department] Ministry of SMEs and Startups
[Project Management (Expert) Organization] Korea Technology and Information Promotion Agency of SMEs
[Research Project Name] Startup Growth Technology Development
[Research Project Name] Development of artificial intelligence platform for tumor treatment/management for medical image based radiotherapy
[Contribution rate] 1/1
[Project Performing Organization] Oncosoft Co., Ltd.
[Research Period] Dec. 1, 2020 to Nov. 30, 2022

1. A user customized medical image segmentation model generating method performed by a processor of a medical image model generating apparatus, comprising the steps of:
providing a model generation interface including a segmentation model selection list to predict a target area;
determining a segmentation model selected by a user through the model generation interface as a learning model;
acquiring a medical image to be input to the learning model and learning data for the target area specified in the medical image; and
generating the segmentation model configured to predict a different target area by inputting a 3D medical image by retraining the learning model based on the medical image again.

2. The user customized medical image segmentation model generating method according to claim 1, further comprising:
after the step of generating the segmentation model,
predicting a plurality of target areas by inputting the 3D medical image including at least two or more target areas to the generated segmentation model and a previously stored segmentation model.

3. The user customized medical image segmentation model generating method according to claim 1, further comprising:
after the step of generating the segmentation model,
a step of acquiring a prediction data based on a previously stored medical image using the generated segmentation model; and
a step of verifying the generated segmentation model by comparing the prediction data and the learning data.

4. The user customized medical image segmentation model generating method according to claim 3, further comprising:
after the step of verifying the segmentation model,
a step of providing a model usage interface including a search region to specify at least one of a subject condition, the target area, and a type of the segmentation model predicting the target area.

5. The user customized medical image segmentation model generating method according to claim 1, wherein the step of determining as the learning model further includes:
a step of providing a learner adjusting region to adjust at least one of hyper parameters, a configuration, an image coordinate system of input data, and trained weights of the learning model, by means of the model generation interface.

6. The user customized medical image segmentation model generating method according to claim 1, further comprising:
after the step of acquiring a medical image,
a step of extracting a name for the target area matched to the learning data; and
a step of labeling a name extracted based on a tag list for every previously stored target area with a new learnable name.

7. The user customized medical image segmentation model generating method according to claim 6, wherein the step of generating the segmentation model further includes:
a step of inputting a labeled medical image to the learning model.

8. The user customized medical image segmentation model generating method according to claim 1, further comprising:
after the step of acquiring the learning data,
a step of generating a plurality of sub volume data using the medical image,
wherein the segmentation model is a model trained to determine a region corresponding to the target area with the plurality of sub volume data as an input.

9. The user customized medical image segmentation model generating method according to claim 8, wherein the sub volume data includes data regarding a moving direction of voxels configuring the sub volume data with respect to any one axis.

10. A user customized medical image segmentation model generating server, comprising:
a communication interface;
a memory; and
a processor operably connected to the communication interface and the memory,
wherein the processor is configured to provide a model generation interface including a segmentation model selection list to predict a target area, determine a segmentation model selected by a user through the model generation interface as a learning model, acquire a medical image to be input to the learning model and learning data for the target area specified in the medical image, and generate a segmentation model configured to predict a different target area by inputting a 3D medical image by retraining the learning model based on the medical image again.

11. The user customized medical image segmentation model generating server according to claim 10, wherein the processor is further configured to after generating a segmentation model, predict a plurality of target areas by inputting the 3D medical image including at least two or more target areas to the generated segmentation model and a previously stored segmentation model.

12. The user customized medical image segmentation model generating server according to claim 10, wherein the processor is further configured to after generating a segmentation model, acquire a prediction data based on a previously stored medical image using the generated segmentation model and verify the generated segmentation model by comparing the prediction data and the learning data.

13. The user customized medical image segmentation model generating server according to claim 12, wherein the processor is further configured to after verifying the segmentation model, provide a model usage interface including a search region to specify at least one of a subject condition, the target area, and a type of the segmentation model predicting the target area.

14. The user customized medical image segmentation model generating server according to claim 10, wherein the processor is further configured to provide a learner adjusting region to adjust at least one of hyper parameters, a configuration, an image coordinate system of input data, and trained weights of the learning model, by means of the model generation interface.

15. The user customized medical image segmentation model generating server according to claim 10, wherein the processor is further configured to after acquiring the medical image, extract a name for the target area matched to the learning data and label a name extracted based on a tag list for every previously stored target area with a new learnable name.

16. The user customized medical image segmentation model generating server according to claim 15, wherein the processor is further configured to input a labeled medical image to the learning model.

17. The user customized medical image segmentation model generating server according to claim 10, wherein the processor is further configured to after acquiring the learning data, generate a plurality of sub volume data using the medical image, and the segmentation model is a model trained to determine a region corresponding to the target area with the plurality of sub volume data as an input.

18. The user customized medical image segmentation model generating server according to claim 17, wherein the sub volume data includes data regarding a moving direction of voxels configuring the sub volume data with respect to any one axis.
